(19) United States
(12) Patent Application Publication
(10) Pub. No.: US 2023/0367999 A1
(43) Pub. Date: Nov. 16, 2023

(54) MULTI-PARAMETER ACCURATE PREDICTION METHOD AND SYSTEM FOR THREE-DIMENSIONAL TIME-SPACE SEQUENCE OF SEAWATER QUALITY

(71) Applicant: Guangdong Ocean University, Zhanjiang (CN)

(72) Inventors: Ji WANG, Zhanjiang (CN); Wenjing LIU, Zhanjiang (CN); Zaimi XIE, Zhanjiang (CN); Xiaoli REN, Zhanjiang (CN)

(21) Appl. No.: 18/357,279

(22) Filed: Jul. 24, 2023

Related U.S. Application Data
(63) Continuation of application No. PCT/CN2023/088258, filed on Apr. 14, 2023.

(30) Foreign Application Priority Data
Apr. 19, 2022 (CN) 202210410548.8

(51) Int. Cl. G06N 3/0455; G06N 3/063; G06N 3/0464

(52) U.S. Cl. CPC G06N 3/0455; G06N 3/063; G06N 3/0464

(57) ABSTRACT

Provided are a multi-parameter accurate prediction method and a system for three-dimensional time-space sequence of seawater quality, which includes the following steps: obtaining key parameters of the seawater quality, and processing the key parameters to obtain target key parameters; obtaining time-space feature information among the target key parameters based on space attention; obtaining predicted future data sequence information based on time attention and the time-space feature information; predicting future water quality multi-parameter contents based on the time-space feature information and the predicted future data sequence information to obtain prediction results.

### CROSS-REFERENCE TO RELATED APPLICATIONS

This application is a continuation of PCT/CN2023/088258, filed on Apr. 14, 2023 and claims priority to Chinese Patent Application No. 202210410548.8, filed on Apr. 19, 2022, the entire contents of which are incorporated herein by reference.

### TECHNICAL FIELD

The application belongs to the field of seawater quality parameter prediction, and in particular to a multi-parameter accurate prediction method and a system for three-dimensional time-space sequence of seawater quality.

### BACKGROUND

With the recent developments in marine technology, data could be used to summarize the laws of nature and society, and predict future trends. Big data is fully utilized to help human beings cope with the climate change, protect the ecological environment and prevent natural disasters. However, the multi-parameter accurate prediction of time-space sequence of the seawater quality has always been a major issue for marine researchers. In order to solve this problem, scholars have applied the machine learning technology to predict the key parameters of aquaculture water quality, which has aroused widespread interest in academia and industry.

With the rise of machine learning, machine learning algorithms are increasingly widely used in accurately predicting aquaculture water quality (lakes, ponds and seawater), especially in accurately predicting seawater quality. Y. Chen et al. put forward Silhouette Coefficient (SC)-K-means-Radial Basis Function (RBF) prediction model to predict the dissolved oxygen content of three-dimensional space sequence of water quality, and the prediction accuracy is 93%. The combination of SC-K-means may denoise the data, RBF may overcome the training local minimum and eliminate data redundancy and errors, and the single parameter of water quality of three-dimensional space sequence is considered, but this model is only used for short-term time sequence and single parameter prediction. The increasingly developed deep learning may learn short-term time sequence water quality data well. Therefore, the Long Short-Term Memory (LSTM) prediction model was constructed by Z. Hu and Y. Liu, aiming to predict the water quality. The LSTM network has advantages of forgetting gate and updating gate processing features, and handle long-term time sequence water quality data well. J. Xie et al. used the Gate Recurrent Unit) (GRU) network with fewer parameters and higher efficiency than those of LSTM and RNN network to build an Attention-GED (GRU encoder-decoder) model to predict the sea surface temperature in a large-scale and different time sequence, thus solving the problem of predicting water quality parameters in different time sequence.

In view of the problems existing in the research on accurately predicting seawater quality by scholars using the machine learning technology, the following aspects are discussed:
\textcircled{1} Engineering in the field of aquaculture water quality
(1) Prediction of single parameters of pond water quality of short-term time sequence and three-dimensional space.
(2) Single parameter or double parameter prediction of seawater quality in long-term and short-term time sequence.
(3) Single parameter prediction of pond water quality in long-term and short-term time sequence and space sequence.
\textcircled{2} Application of the deep learning technology in seawater quality prediction
(1) Using the model of combining time-space attention with GED (Attention-GED)
(2) Using the model of combining Convolutional Neural Network (CNN) with LSTM (ConvLSTM)

To sum up, firstly, in the field of seawater, scholars have not considered the multi-parameter prediction of seawater, in order to determine the quality in both long-term and short-term sequence and three-dimensional space sequence. Secondly, existing methods have not considered the correlation among the multi-parameter features of seawater quality extracted by fusion data processing algorithm, time-space attention, CNN and GED methods.

### SUMMARY

In order to make up for the shortcomings of the above scholars in the study on predicting and applying seawater, accurately predict multi-parameter of seawater quality, explore the relationship among the multi-parameter features, and study the use of the deep learning technology to improve the multi-parameter prediction accuracy of water quality. The application provides a method for accurately predicting multi-parameters of three-dimensional time-space sequence of seawater quality, including the following steps:
obtaining key parameters of the seawater quality, and processing the key parameters to obtain target key parameters;
obtaining time-space feature information among the target key parameters based on space attention;
obtaining predicted future data sequence information based on time attention and the time-space feature information;
predicting future water quality multi-parameter contents based on the time-space feature information and the predicted future data sequence information to obtain prediction results.

Optionally, the process of processing the key parameters to obtain the target key parameters includes: carrying out a noise reduction processing on the key parameters to obtain key parameter components; inputting the key parameter components into a CNN network, and extracting time-space features among the key parameter components.

Optionally, the process of carrying out the noise reduction processing on the key parameters includes decomposing the key parameters into subsequences and residual sequences, and performing a combination of random components, trend components and detail components by using a sample entropy algorithm.

Optionally, the process of obtaining the time-space feature information among the target key parameters based on the space attention includes: dynamically learning the time-space features among the target key parameters to obtain a first weight based on the space attention; inputting time-space features into a GRU encoder network to obtain a first hidden state; and obtaining the time-space feature information among the target key parameters based on the first weight and the first hidden state;

Optionally, the process of obtaining the predicted future data sequence information based on the time attention and the time-space feature information includes: processing the time-space feature information with the time attention to obtain a second weight; inputting the time-space feature information into the GRU encoder network to obtain a second hidden state; obtaining the predicted future data sequence information based on the second weight and the second hidden state;

Optionally, the process of predicting the future water quality multi-parameter contents based on the time-space feature information and the predicted future data sequence information includes: inputting the time-space feature information and the predicted future data sequence information into the GRU encoder network for encoding to convert into a fixed-length vector; decoding the fixed-length vector, converting the fixed-length vector into an output sequence, and predicting the future water quality multi-parameter contents.

The application also provides a system for accurately predicting multi-parameters of three-dimensional time-space sequence of seawater quality, which includes:
a parameter obtaining module, used for obtaining key parameters of seawater quality;
a parameter processing module, connected with the parameter obtaining module and used for processing the key parameters to obtain target key parameters;
an attention algorithm module, used for obtaining time-space feature information and predicted future data sequence information among the target key parameters; and
a predicting module, used for predicting future water quality multi-parameter contents according to the time-space feature information and the predicted future data sequence information to obtain prediction results.

Optionally, the parameter processing module includes a noise reduction processing unit and a feature extracting unit;
the noise reduction processing unit is used for performing a noise reduction processing on the key parameters to obtain key parameter components; and
the feature extracting unit is used for extracting time-space features among the key parameter components through a CNN network.

Optionally, the attention algorithm module includes a space attention unit, wherein the space attention unit includes a first weight unit, a first hidden state unit and a first information obtaining unit;
the first weight unit is used for dynamically learning time-space features among the target key parameters through space attention to obtain a first weight;
the first hidden state unit is used for obtaining a first hidden state through a GRU encoder network;
the first information obtaining unit is used for obtaining the time-space feature information among the target key parameters according to the first weight and the first hidden state.

Optionally, the attention algorithm module includes a time attention unit, wherein the time attention unit includes a second weight unit, a second hidden state unit and a second information obtaining unit;
the second weight unit is used for processing the time-space feature information through time attention to obtain a second weight;
the second hidden state unit is used for obtaining a second hidden state through the GRU encoder network;
the second information obtaining unit is used for obtaining the predicted future data sequence information according to the second weight and the second hidden state.

The application discloses the following technical effects.

The method for accurately predicting multi-parameters of three-dimensional time-space sequence of seawater quality provided by the application may improve the extraction rate of multi-parameter feature information of seawater quality of time sequence and space sequence, reduce the non-stationarity of multi-parameter data of seawater quality, and improve the prediction accuracy of water quality time sequence and three-dimensional space multi-parameters.

### BRIEF DESCRIPTION OF THE DRAWING

In order to explain the embodiments of the present application or the technical scheme in the prior art more clearly, the drawings needed in the embodiments will be briefly introduced below. Obviously, the drawings described below are only some embodiments of the present application, and other drawings may be obtained according to these drawings without creative work for ordinary technicians in the field.

FIG. 1 is a method flow chart of the embodiment in the present application.

### DETAILED DESCRIPTION OF THE EMBODIMENTS

In the following, the technical scheme in the embodiment of the application will be clearly and completely described with a reference to the attached drawings. Obviously, the described embodiment is only a part of the embodiment of the application, but not the whole embodiment. Based on the embodiments in the present application, all other embodiments obtained by ordinary technicians in the field without creative labor will fall in the scope of protection of the present application.

In order to make the above objectives, features and advantages of the present application more obvious and easier to understand, the present application will be further described in detail with the attached drawings and specific embodiments.

### Embodiment 1

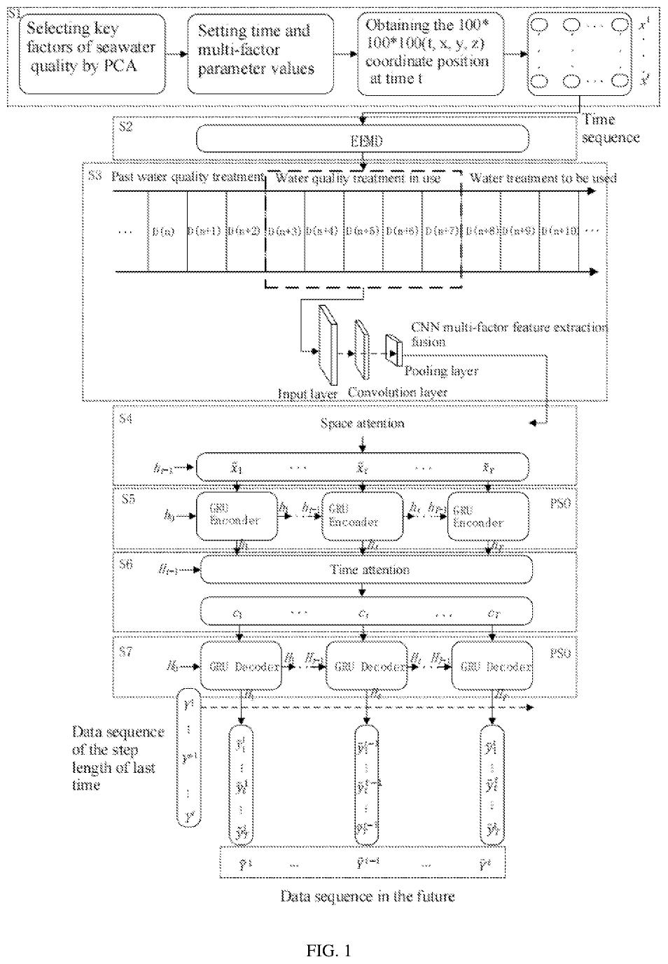

As shown in FIG. 1, the application provides a method for accurately predicting multi-parameters of three-dimensional time-space sequence of seawater quality, which includes the following steps.
S1, the key parameters of seawater quality are optimized by PCA algorithm and the correlation coefficients are calculated; each key parameter data X^{k} (k=1, 2, . . . , t) is a four-dimensional vector, where X=[x₁, x₂, x₃, x₄]^{T}, x₁ is the sampling time, and x₂, x₃, x₄ are the three-dimensional coordinates of sampling points in seawater area; the key parameters of the seawater quality are evenly distributed on the vertical coordinates; when time t is set, the 50×50×50(t, x, y, z) coordinate position of each key parameter at time t is generated;
S2, EEMD is used to carry out the noise reduction processing on the optimized key parameters of water quality; EEMD decomposes the original sequence of all key parameters, calculates the correlation features, and decomposes the original sequence into x natural modal components with different features, IMF1-IMFx and a residual component RES; then, the sample entropy of the sub-sequence after the decomposition of each key parameter of water quality is calculated to combine components, and after judgment to reorganize into random components, trend components and detail components, that is, each IMF component is superimposed;
S3, the random components, trend components and detail components of the key parameters of d time sequence are selected through sliding window and entered into the input layer of the CNN network for processing, and the convolution layer and pooling layer extract features among all key parameter components respectively;
S4, the space attention is used to dynamically learn the space features among key parameters of water quality, and the generated weights are \tilde{x}₁, \tilde{x}₂, . . . , and \tilde{x}_{T};
S5, the space features among the extracted key parameters of water quality are input into the GRU encoder, and the GRU encoder inputs the previous hidden state hₜ₋₁ or h₀ and historical water quality data (that is, the past water quality sequence) in each time step, and a new hidden state Hᵢ (i=1, 2, . . . , k) will be generated in each time step; after all the historical sequences are processed by the model, the hidden states h₁, h₂, . . . , hₖ are generated, and each hidden state corresponds to the weights \tilde{x}₁, \tilde{x}₂, . . . , and \tilde{x}_{T} generated by the space attention; the feature information among all the key parameters of water quality is obtained by calculating the hidden states hᵢ (i=1, 2, . . . , k) and \tilde{x}ⱼ (j=1, 2, . . . , T);
S6, the data of each historical sequence has different influences on the future data prediction. Therefore, all the time sequence hᵢ\tilde{x}ⱼ (i=1, 2, . . . , k; j=1, 2, . . . , T) are input into the time attention to learn the influence of the hidden state of the GRU decoder network in each time window, and then the weights c₁, c₂, . . . , c_{T} are generated. In each time step, a new hidden state will be generated; after processing the historical data, H₁, H₂, . . . , H_{T} will be generated to correspond to c₁, c₂, . . . , c_{T}, and all the predicted future data sequence information is obtained by calculating hᵢ (i=1, 2, . . . , k) and Cⱼ (j=1, 2, . . . , T);
S7, Hᵢcⱼ (i=1, 2, . . . , k; j=1, 2, . . . , t) is used to combine with the key parameter content sequence in the previous step and input to the GRU decoder to predict the future water quality multi-parameter contents; the network is very flexible for multi-scale parameter prediction, and the hidden state size is the same as the coding.

### Embodiment 2

The application also provides a system for accurately predicting multi-parameters of three-dimensional time-space sequence of seawater quality, which includes:
a parameter obtaining module, used for obtaining the key parameters of seawater quality and reducing the interference of other physical or water quality factors which have little correlation with the key parameters of seawater quality;
the parameter obtaining module includes a PCA algorithm unit and an improved EMD algorithm unit; the PCA algorithm unit is used to optimize the key parameters of seawater quality and reduce the interference of other physical or water quality factors which have little correlation with the key parameters of seawater quality; the improved EMD algorithm unit is used to reduce the non-stationarity of key parameters of seawater quality;
a parameter processing module, connected with the parameter obtaining module and used for processing the key parameters to obtain the target key parameters; the key parameters of seawater quality include PH value, ammonia nitrogen, total phosphorus, dissolved oxygen and chemical oxygen demand, and the prediction sequence is time sequence and three-dimensional space sequence;
the parameter processing module includes a noise reduction processing unit and a feature extracting unit; the noise reduction processing unit is used for performing the noise reduction processing on the key parameters to reduce the non-stationarity of the key parameters of seawater quality; the feature extracting unit is used for extracting the time-space features among the key parameter components through the CNN network;
an attention algorithm module is used for obtaining the time-space feature information among the target key parameters and predicting the future data sequence information; the attention algorithm module includes a time attention unit and a space attention unit; the space attention is used to dynamically learn the space correlation between external attributes, and the time attention is used to learn the influence of the hidden state of GRU encoder network in each time window; the external attributes are the key parameters of seawater quality;
the space attention unit is used for dynamically learning the space correlation between the external attributes, and the external attributes are key parameters of seawater quality; the space attention unit includes a first weight unit, a first hidden state unit and a first information obtaining unit; the first weight unit is used for dynamically learning the time-space features among the target key parameters through the space attention to obtain a first weight; the first hidden state unit is used for obtaining a first hidden state through the GRU encoder network; the first information obtaining unit is used for obtaining the time-space feature information among the target key parameters according to the first weight and the first hidden state;
the time attention unit is used for learning the influence of the hidden state of the GRU encoder network in each time window; the time attention unit includes a second weight unit, a second hidden state unit and a second information obtaining unit; the second weight unit is used for processing the time-space feature information through the time attention to obtain a second weight; the second hidden state unit is used to obtain a second hidden state through the GRU encoder network; the second information obtaining unit is used for obtaining the predicted future data sequence information according to the second weight and the second hidden state;
and a predicting module is used for predicting the future water quality multi-parameter contents according to the time-space feature information and the predicted future data sequence information, and obtaining prediction results.

This method makes up for the shortcomings of the existing technology in the research of seawater prediction and application, and puts forward the deep learning model to predict the multi-parameters of seawater quality (more than 3 parameters) of the long-term and short-term sequence and three-dimensional space. On the basis of the existing research results of the scholars, the improvement of EMD algorithm (EEMD) and the integration of time-space attention, CNN and GED network may carry out the noise reduction processing on the data and extract the features among multi-parameters, which may improve the multi-parameter prediction accuracy of water quality.

The method for accurately predicting multi-parameters of three-dimensional time-space sequence of seawater quality provided by the application may improve the extraction rate of multi-parameter feature information of seawater quality of time sequence and space sequence, reduce the non-stationarity of multi-parameter data of seawater quality, and improve the prediction accuracy of water quality time sequence and three-dimensional space multi-parameters.

What is described in the embodiments in this specification is only an enumeration of the realization forms of the inventive concept, and the scope of protection of the application should not be regarded as limited to the specific forms stated in the examples, and the scope of protection of the application also covers equivalent technical means that may be thought of by those skilled in the art according to the inventive concept.

What is claimed is:

1. A method for accurately predicting multi-parameters of three-dimensional time-space sequence of seawater quality, comprising:
obtaining key parameters of the seawater quality, and processing the key parameters to obtain target key parameters;
obtaining time-space feature information among the target key parameters based on space attention;
obtaining predicted future data sequence information based on time attention and the time-space feature information; and
predicting future water quality multi-parameter contents based on the time-space feature information and the predicted future data sequence information to obtain prediction results;
wherein a process of obtaining the time-space feature information among the target key parameters based on the space attention comprises: dynamically learning the time-space features among the target key parameters based on the space attention to obtain a first weight; inputting the time-space features into a GRU encoder network to obtain a first hidden state; and obtaining the time-space feature information among the target key parameters based on the first weight and the first hidden state;
a process of obtaining the predicted future data sequence information based on the time attention and the time-space feature information comprises: processing the time-space feature information with the time attention to obtain a second weight; inputting the time-space feature information into the GRU encoder network to obtain a second hidden state; and obtaining the predicted future data sequence information based on the second weight and the second hidden state; and
a process of predicting the future water quality multi-parameter contents based on the time-space feature information and the predicted future data sequence information comprises: inputting the time-space feature information and the predicted future data sequence information into the GRU encoder network for encoding to convert into a fixed-length vector; decoding the fixed-length vector, converting the fixed-length vector into an output sequence, and predicting the future water quality multi-parameter contents.

2. The method for accurately predicting multi-parameters of three-dimensional time-space sequence of seawater quality according to claim 1, wherein:
a process of processing the key parameters to obtain the target key parameters comprises: carrying out a noise reduction processing on the key parameters to obtain key parameter components; inputting the key parameter components into a CNN network, and extracting the time-space features among the key parameter components.

3. The method for accurately predicting multi-parameters of three-dimensional time-space sequence of seawater quality according to claim 2, wherein:
a process of carrying out the noise reduction processing on the key parameters comprises: decomposing the key parameters into subsequences and residual sequences, and performing a combination of random components, trend components and detail components by using a sample entropy algorithm.

4. A system for accurately predicting multi-parameters of three-dimensional time-space sequence of seawater quality, comprising:
a parameter obtaining module, used for obtaining key parameters of seawater quality;
a parameter processing module, connected with the parameter obtaining module and used for processing the key parameters to obtain target key parameters;
an attention algorithm module, used for obtaining time-space feature information and predicted future data sequence information among the target key parameters; and
a predicting module, used for predicting future water quality multi-parameter contents according to the time-space feature information and the predicted future data sequence information to obtain prediction results;
wherein the attention algorithm module comprises a space attention unit, and the space attention unit comprises a first weight unit, a first hidden state unit and a first information obtaining unit;
the first weight unit is used for dynamically learning time-space features among the target key parameters through space attention to obtain a first weight;
the first hidden state unit is used for obtaining a first hidden state through a GRU encoder network;
the first information obtaining unit is used for obtaining the time-space feature information among the target key parameters according to the first weight and the first hidden state;
the attention algorithm module comprises a time attention unit, wherein the time attention unit comprises a second weight unit, a second hidden state unit and a second information obtaining unit;
the second weight unit is used for processing the time-space feature information through time attention to obtain a second weight;
the second hidden state unit is used for obtaining a second hidden state through the GRU encoder network;
the second information obtaining unit is used for obtaining the predicted future data sequence information according to the second weight and the second hidden state; and
a process of predicting the future water quality multi-parameter contents based on the time-space feature information and the predicted future data sequence information comprises: inputting the time-space feature information and the predicted future data sequence information into the GRU encoder network for encoding to convert into a fixed-length vector; decoding the fixed-length vector, converting the fixed-length vector into an output sequence, and predicting the future water quality multi-parameter contents.

5. The system for accurately predicting multi-parameters of three-dimensional time-space sequence of seawater quality according to claim 4, wherein,
the parameter processing module comprises a noise reduction processing unit and a feature extracting unit;
the noise reduction processing unit is used for performing a noise reduction processing on the key parameters to obtain key parameter components; and
the feature extracting unit is used for extracting time-space features among the key parameter components through a CNN network.
